(19) United States
(12) Patent Application Publication
(10) Pub. No.: US 2015/0301019 A1
(43) Pub. Date: Oct. 22, 2015

(54) Hand Held Breath Analyzer

(71) Applicant: James R. Smith, Boulder, CO (US)

(72) Inventor: James R. Smith, Boulder, CO (US)

(21) Appl. No.: 14/254,415

(22) Filed: Apr. 16, 2014

(51) Int. Cl. G01N 33/497

(52) U.S. Cl. CPC G01N 33/497

(57) ABSTRACT

A portable breath analyzer is described including a housing that encloses a probe assembly with two probes: one responsive to the 12CO2 isotopes in a breath sample, and the other responsive to 13CO2 isotopes. Each probe includes a sample cell containing exhaled breath, a correlation cell containing a selected one of the isotopes, and a calibration cell. An IR energy source is associated with each probe. Each IR source causes propagation of infrared energy through the associated sample cell, and into the correlation cell. Gas sample probes may be aligned in series or parallel and respective correlation cells are modified to accommodate the selected probe configuration. MEMS pressure transducers may be utilized in a common wall between adjacent correlation cells to thereby sense a pressure differential caused by the absorption of pulsed IR energy in the correlation cells and to directly indicate an isotopic ratio. A MEMS transducer positioned between adjacent calibration cells may also generate a signal that is utilized to compensate for any difference in IR energy source intensity.

### CROSS-REFERENCE TO RELATED APPLICATIONS

Not Applicable

### FEDERAL SPONSORSHIP

Not Applicable

### JOINT RESEARCH AGREEMENT

Not Applicable

### TECHNICAL FIELD

This invention pertains generally to instruments used to analyze gas mixtures. More particularly, this invention pertains to portable devices sensitive to the presence of gas mixtures in exhaled air that may be used to analyze the exhaled air for the presence or absence of a targeted gas mixture.

### BACKGROUND

Generally, it is known that the CO2 in exhaled air of humans includes naturally occurring levels of 13CO2 and 12CO2 isotopes. For example, a human breath may contain approximately 3% CO2 by volume or approximately 30,000 ppm and this volume of CO2 may contain approximately 1% 13CO2 isotopes or 300 ppm. It is also known that inhaled air includes a background atmospheric concentration of 12CO2 isotope of approximately 400 ppm. The 12CO2 isotope present in the background atmospheric concentration is detectable within 0.1 ppm. The 12CO2 and 13CO2 isotopes have previously been detectable and distinguished from a human breath. In the past, bulk mass-spectroscopic equipment has been utilized in attempts to determine and resolve, to a high precision, separation of the 12CO2 and 13CO2 isotopes.

The detection of increased 13CO2 may be used advantageously in conjunction with a breath test to diagnose the presence of gastrointestinal pathogens in a patient. For example, it is known that urease breaks down urea (CO(NH2)2) into ammonia and CO2 and it is also known that gastrointestinal pathogens produce urease. When urease is present in a patient infected with a gastrointestinal pathogen, orally administered urea will be broken down by the urease to produce ammonia and CO2. Further, ingested urea labeled with carbon-13 may be utilized to detect an increase of 13CO2 and the presence of a urease producing gastrointestinal pathogen. When urease is present the amount of discerned 13CO2 increases after urea is ingested.

Gastric infection with Helicobacter pylori (H. pylori) is widely recognized as the primary cause of gastritis and is believed to be a contributor or cause of many duodenal ulcers, gastric ulcers, or gastric cancer. The gastrointestinal pathogen H. pylori produces urease that is detectable to diagnose the presence of this pathogen. Thus, increased levels of expressed CO2 having the labeled 13CO2 indicate the presence of unwanted bacteria within a human's digestive system. Once detected, treatment of the infection with antimicrobial therapy is relatively inexpensive and frequently successful. However, in the past, discerning increases in the levels of 13CO2 and diagnosis of the infection has been expensive. Additionally, other detection methods, such as endoscopy and gastric biopsy require less desirable invasive procedures. Also, other prior methods are not particularly useful to test for successful treatment of the infection. Hence, the ability to discern gas mixtures with nonintrusive, mobile, cost effective equipment is desirable.

### SUMMARY

Embodiments according to aspects of the invention include an apparatus and method for detecting gas mixtures in a sample. A device of the invention includes an air intake, sample cells adapted to receive an air sample, correlation cells having hermetically sealed gas chambers therein, radiant energy sources, an isotopic analyzer, an air outtake, and air conduits coupling the air intake, sample cells and air outtake. The correlation cells include a first correlation cell having 12CO2 isotopes of carbon dioxide gas and a second correlation cell having 13CO2 isotopes of carbon dioxide gas. A housing may contain the sample cells, correlation cells, radiant energy sources, isotopic analyzer and air conduits. In an embodiment of the invention the correlation cells are bi-directional. Also, in an embodiment of the invention the sample cells and correlation cells may be aligned in series. Also described herein is a radiant energy source that includes a single radiant energy generator and a beam splitter that directs radiant energy towards separate sample cells and correlation cells. The radiant energy source may include collimating optics and band pass filters coupled with the radiant energy sources to transmit radiant energy at selected bandwidths that are absorbs by known select gases. The device may also include a valve, flow meter, and pumps coupled to the air conduits to purge the sample cells.

In an embodiment of the invention there is provided a device for determining relative concentrations of a plurality of isotopes of a gas in a gas sample. The device includes a first sample cell adapted to receive a first portion of a gas sample comprising a selected gas, and a second sample cell adapted to receive a second portion of the same gas sample. The device includes air conduits that split the sample of gas, directing a portion of the gas sample to a first sample cell used to determine response to 13CO2 and directs another portion of the gas sample to a second sample cell used to determine the response to 12CO2. A first correlation cell contains a first gas comprising 13CO2 isotopes of the selected gas while being substantially free of 12CO2 isotopes of the selected gas. A second correlation cell contains a second gas (for example 002) comprising the 12CO2 isotope while being substantially free of the 13CO2 isotope. A radiant energy source is collimated to direct pulsed radiant energy along a first path through the first sample cell and into the first correlation cell.

In embodiments of the invention the device includes two radiation sources each source associated with corresponding sample cell and correlation cell. Alternatively, there may be ways of using a single radiation source to illuminate both sample cells. If two radiation sources are used, then there must be some mechanism to calibrate them to the same precise scale. A single radiation source requires that its radiation be split and directed into the two sample cells. In both cases operation of the device resolves logically to illumination of the two correlation cells by a single radiation source.

In an embodiment of the invention a pulsed source of radiation is collimated and transmitted along a first path through a first band pass filter, the first sample cell and a bi-directional correlation cell containing significant concentrations of both isotopes. A second pulsed source is collimated along a second path through a second band pass filter, the second sample cell and into the same bi-directional correlation cell from the opposite direction. A sensing component, operatively associated with the correlation cell, is responsive to absorption of radiant energy from either direction in the correlation cell by the first gas at a first absorption level and further is responsive to absorption of radiant energy in the correlation cell by the second gas at a second absorption level. The sensing component is adapted to phasing of the activation of the radiation sources to thereby compare the first and second absorption levels and to generate a measure of the ratio of concentrations of the two isotopes in the gas sample.

In an embodiment of the invention, an additional bi-directional correlation cell (calibration cell) is situated in the device adjacent the correlation cell containing targeted isotopes. The calibration bi-directional correlation cell contains a pure sample of a selected gas that has absorption of radiant energy per molecule comparable to that of either isotope. Comparison of the signals from the calibration cell may be the sole or alternative method to calibrate the ratio of the radiation streams incident on the sample cells.

In an embodiment of the invention a single source of radiant energy is utilized, with a stream of radiation split in two and then transmitted into a pair of probes. Each probe includes a radiation filter, sample cell and correlation cell. A discrete sensing component, operatively associated with each correlation cell, is responsive to absorption of radiant energy in the correlation cell by the first isotope at a first absorption level and further is responsive to absorption of radiant energy in the correlation cell by the second isotope at a second absorption level.

A device is described, wherein the sensing component is a pressure transducer adapted to detect a difference in pressure between the two halves of the bi-directional correlation cell or the fluctuating pressure of the optically active volume of the correlation cell. A ratio of the changes in pressure, when corrected for the response to zero isotopic concentrations in the sample cells, may be utilized to determine a ratio of concentrations of the select isotopes.

In devices of the invention detecting changes in pressure, the difference in pressure is a direct result of the change in the absorption of radiation in the correlation cells. In each correlation cell, absorption of photons by the gas molecules momentarily increases the gas temperature, causing a corresponding increase in gas pressure. A difference in pressure between the correlation cells reflects a difference in radiant energy absorption within the cells. Consequently, the isotopic ratio is determined using relatively low cost pressure transducers in lieu of the expensive photoelectric detectors.

A further aspect of the present invention is a calibration protocol for determining relative concentrations of isotopes of a gas in a breath sample. The calibration protocol includes directing pulsed radiant energy along a straight path containing a first radiation filter, a first portion of a breath sample and a first correlation cell or the first half of a bi-directional polar correlation cell; directing pulsed radiant energy along a second straight path through a second radiation filter, a second portion of the breath sample and a second correlation cell or the second half of a bi-directional correlation cell; correcting signals from the correlation cells for the responses to zero CO2 concentrations in the sample cells by scheduled or on-demand application of air stripped of CO2 by an internal mechanism or by signals from an internal calibration cell; and expressing the ratio of the corrected signals as the means to generate the desired measure of the ratio of concentrations of the first and second isotopes in the breath sample.

Another aspect of the invention is a portable breath analyzer. The portable device includes a first sample cell adapted to receive and contain a first portion of a breath sample, and a second sample cell adapted to receive and contain a second portion of the breath sample. A first correlation cell contains a first gas that comprises a first isotope of a selected gas while being substantially free of a second isotope of the selected gas. A second correlation cell contains a second gas that comprises the second isotope of the selected gas while being substantially free of the first isotope. A radiant energy source is adapted to direct pulsed radiant energy along a first path through the first sample cell and into the first correlation cell, and further is adapted to direct pulsed radiant energy along a second path through the second sample cell and into the second correlation cell. A sensing component, operatively associated with the first and second correlation cells, is adapted to compare a first level of absorption of radiant energy by the first gas in the first correlation cell with a second level of absorption of the radiant energy by the second gas in the second correlation cell, to generate an indication of relative concentration of the first isotope and the second isotope in the breath sample.

Thus in accordance with the present invention, a low cost, portable instrument is capable of generating accurate, real time indications of isotopic ratios in exhaled air and other gasses. The analyzer is convenient and safe for the patient or test subject, due to a convenient, disposable interface. The analyzer is easy for a physician or other user to operate, and it can be used in successive tests without any intervening adjustments or resetting.

The accompanying drawings, which are incorporated in and constitute a portion of this specification, illustrate embodiments of the invention and, together with the detailed description, serve to further explain the invention. The embodiments illustrated herein are presently preferred; however, it should be understood, that the invention is not limited to the precise arrangements and instrumentalities shown. For a fuller understanding of the nature and advantages of the invention, reference should be made to the detailed description in conjunction with the accompanying drawings.

### DESCRIPTION OF THE DRAWINGS

In the various figures, which are not necessarily drawn to scale, like numerals throughout the figures identify substantially similar components. Further, although the sectional views may be cross hatched to indicate a particular material the cross hatching should not be construed as limiting the component to the particular material designated by the cross hatching.

FIG. 1 is a perspective view of a portable air analyzing device constructed in accordance with the present invention;

FIG. 2 is a schematic view illustrating the flow of air into and through an analyzing device of the present invention;

FIG. 3 is a schematic view illustrating the flow of air into and through an analyzing device of the present invention;

FIG. 4 is an enlarged partial sectional view of a bidirectional correlation cell of the present invention;

FIG. 5 is a sectional view taken along the line 5-5 in FIG. 4;

FIG. 6 is an electrical schematic view of components of an analyzing device in accordance with present invention;

FIG. 7 is a schematic view illustrating the flow of air into and through an analyzing device of the present invention;

FIG. 8 is a schematic view illustrating the flow of air into and through an analyzing device of the present invention;

FIG. 9 is an enlarged partial sectional view of an alternate correlation cell of the present invention:

FIG. 10 is a sectional view taken along line 9-9 in FIG. 9;

FIG. 11 is an electrical schematic view of components of an analyzing device in accordance with the present invention;

FIG. 12 is a schematic view illustrating the flow of air into and through an analyzing device of the present invention;

FIG. 13 is a schematic view illustrating the flow of air into and through an analyzing device of the present invention;

FIG. 14 is an enlarged partial section view of an alternate correlation cell of the present invention;

FIG. 15 is a sectional view taken along line 14-14 in FIG. 14;

FIG. 16 is an enlarged partial sectional view of an alternate correlation cell of the present invention;

FIG. 17 is a sectional view taken along line 16-16 in FIG. 16;

FIG. 18 is an enlarged partial sectional view of an alternate correlation cell of the present invention;

FIG. 19 is an enlarged partial sectional view of an alternate correlation cell of the present invention;

FIG. 20 is an enlarged partial sectional view of a radiation filter of the present invention;

FIG. 21 is an end view taken along line 21-21 in FIG. 20;

FIG. 22 is an enlarged partial section view of an alternate radiation filter of the present invention;

FIG. 23 is a sectional view taken along line 23-23 in FIG. 22;

FIG. 24 is a perspective view of a portable air analyzing device constructed in accordance with the present invention;

FIG. 25 is a partial sectional view of a portable analyzing device constructed in accordance with the present invention;

FIG. 26 is a partial sectional view of a portable analyzing device constructed in accordance with the present invention; and

FIG. 27 is a partial sectional view of a portable analyzing device constructed in accordance with the present invention.

### DETAILED DESCRIPTION

The following description provides detail of various embodiments of the invention, one or more examples of which are set forth below. Each of these embodiments are provided by way of explanation of the invention, and not intended to be a limitation of the invention. Further, those skilled in the art will appreciate that various modifications and variations may be made in the present invention without departing from the scope or spirit of the invention. By way of example, those skilled in the art will recognize that features illustrated or described as part of one embodiment, may be used in another embodiment to yield a still further embodiment. Thus, it is intended that the present invention also cover such modifications and variations that come within the scope of the appended claims and their equivalents.

The air analyzing device of the present invention advantageously includes a housing containing sample cells, correlation cells, a radiant energy source and a sensing component. The housing contains an internal fluid conduit arrangement accessible outside the housing for conducting the first and second portions of the breath sample to the first and second sample cells. A length of tubing with a fitting at one end for a releasable fluid coupling to the conduit arrangement, and a mouthpiece at the opposite end of the tubing, provide a convenient user interface.

In a preferred version of the analyzing device, the sample cells, radiant energy source and sensors are provided as an assembly of two integral probes, one associated with each sensed isotope. Each probe includes a radiant energy source, a sample cell, an associated correlation cell, and collimating optics for directing the radiant energy in a substantially linear path through the sample cell and into the associated correlation cell. In a preferred probe assembly the probes are linearly arranged back to back with their respective correlation cells adjacent one another. The radiant energy from sources at opposite ends of the probe assembly travels in two opposite directions toward a junction of the correlation cells.

A further aspect of the present invention is a process for determining relative concentrations of a plurality of isotopes of a gas in a breath sample. The process includes:

a. directing pulsed radiant energy along a first path through a first portion of a breath sample and into a first correlation cell containing a first gas, wherein the first gas comprises a first isotope of a selected gas and is substantially free of a second isotope of the selected gas;

b. directing pulsed radiant energy along a second path through a second portion of the breath sample and into a second correlation cell containing a second gas, wherein the second gas comprises the second isotope and is substantially free of the first isotope; and c. sensing a difference in pressure between the first correlation cell and the second correlation cell to generate an indication of relative concentration of the first and second isotopes in the breath sample.

Those skilled in the art will appreciate that the apparatus may be utilized as a diagnostic or detection instrument.

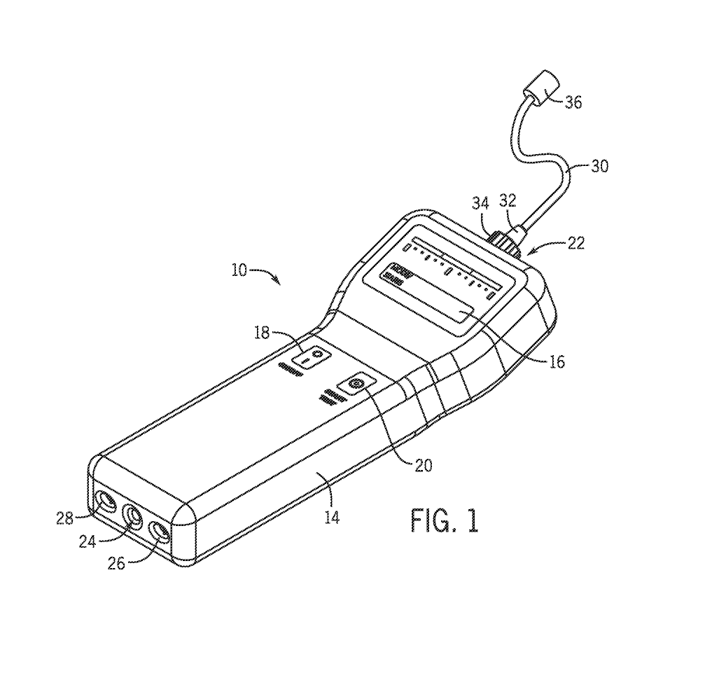

Turning attention now to the Figures, embodiments of the analyzing device or system 10 of the present invention will now be described in more detail. FIG. 1 illustrates a hand held or portable breath analyzer 10 configured to detect relative concentrations of stable isotopes of carbon dioxide in exhaled breath. The device 10 generally includes a housing 14, display 16, power switch 18, start switch 20, gas sample intake 22, gas sample output 24, charging port 26, and docking port 28. The housing 14 may be constructed having a length of less than eight inches, and a width and depth on the order of one fourth to one third the length. In this manner, the device 10 may be constructed to be light weight (of less than or about one pound) and easily carried or manipulated with one hand. The visual display 16 may present results and ratios for the user.

A disposable gas or air intake conduit 30 may be releasably coupled to the gas sample intake 22 via fittings 32 and 34. The fittings may be of a luer lock type. To minimize the entry of moisture and aerosol into the conduit 30 during testing, a mouthpiece including a hydrophobic membrane (formed for example of polytetrafluoroethylene (PTFE)) or desiccant filter 36 may be coupled to a free end of the conduit 30. The membrane or filter 36 preferably would not absorb significant carbon dioxide or differentiate significantly the transmission of isotopes of the gas sample. For example, a molecular sieve with small, three angstrom pores may be appropriate. The interface is disposable and may be used one time per patient or breath test.

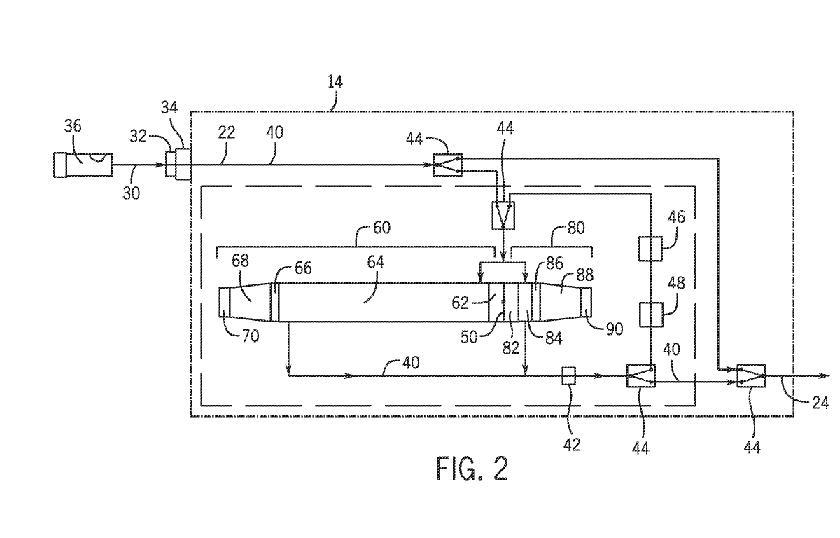

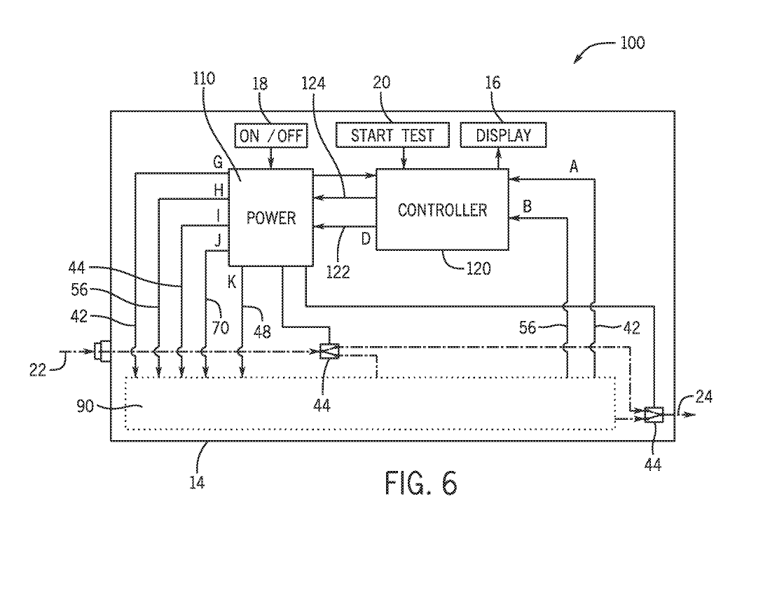

With reference to FIG. 2 a schematic representation of an embodiment of the device 10 is shown illustrating a dual probe with a bi-directional correlation cell 50. The schematic further illustrates a flow of the sample gas through the device. An internal gas conduit 40 directs air through the various internal components of the device (the direction of air travel is represented by arrows), including three way gas valves 44, first sample cell 64, second sample cell 84, flow meter 42, pump 48, scrub unit 46, and gas sample out port 24. Manipulation of the three way valves allow for testing of sample gases and air stripped of both isotopes (zero air). With the three way valves 44 set to conduct air along their solid lines, a gas sample travels from air intake 22, through sample cells 64 and 84, through flow meter 42 and out port 24. With the three way valves 44 set to conduct air along the broken line path, a gas travels from air intake 24 close-cycle through the device. The pump 48 circulates the gas sample, and scrub unit 46 scrubs both isotopes of CO2 to an insignificant level. In this manner, the response to zero air is determined by a controller (see FIG. 6).

The embodiment of the device 10 illustrated in FIG. 2 includes a first probe 60 that includes a correlation cell 62, sample cell 64, band pass filter 66, collimator optics 68, and radiant energy source 70. Likewise, the second probe 80 includes correlation cell 82, sample cell 84, band pass filter 86, collimator optics 88, and radiant energy source 90. First probe 60 is configured to be responsive to 13CO2 and the shorter second probe 80 is configured to be responsive to the higher concentrations of 12CO2. The two probes share a bi-directional correlation cell 50. The collimating optics of both probes promote propagation of radiant energy into respective sample cells in an axial direction. Each probe also includes respective band pass filters 66 and 86 which limits the frequency of radiant energy entering sample cells 64 and 84. The range of the band pass filters are selected to accommodate the targeted isotopes with the result that radiation entering sample cell 64 and correlation cell 62 is stripped of its 12CO2 radiation responsive component, whereas radiation entering sample cell 84 and correlation cell 82 has been stripped of its frequency range responsive to the 13CO2 component. Processing the alternating absorptions in the bi-directional correlation cell 50 (comprised of correlation cell 62 and 82), and corrected for response to zero CO2 in the sample cells, gives the needed measure of the ratio of isotopic concentrations of the sample cells. The preferred infrared sources of radiant energy 70 and 90 are solid state, modulated electronically and intense enough to provide sufficient radiation within the spectral bands of the two isotopes. The sources 70 and 90 can be used to generate selectively pulsed IR energy without the need for chopping or other mechanical modulation.

The bi-directional correlation cell 50 responds to radiant energy from opposing directions by the probes 60 and 80. The radiation sources 70 and 90 may be operated out of phase to selectively measure absorption by the two isotopes of the pair of samples or in phase to null the responses by adjustment of the excitation of one of the radiation sources. Each side 62 and 82 of the bi-directional correlation cell 50 contains significant amounts of both isotopes. Sample cell 64 and 84 have the same diameter, but the lengths of the two cells vary by a factor commensurate with the normal relative concentrations of 12CO2 and 13CO2 in human breath. Specifically, the length of sample cell 64 exceeds the length of sample cell 84 by about two orders of magnitude, to compensate for the weaker absorption (per unit length) by 13CO2 because of its much lower concentration.

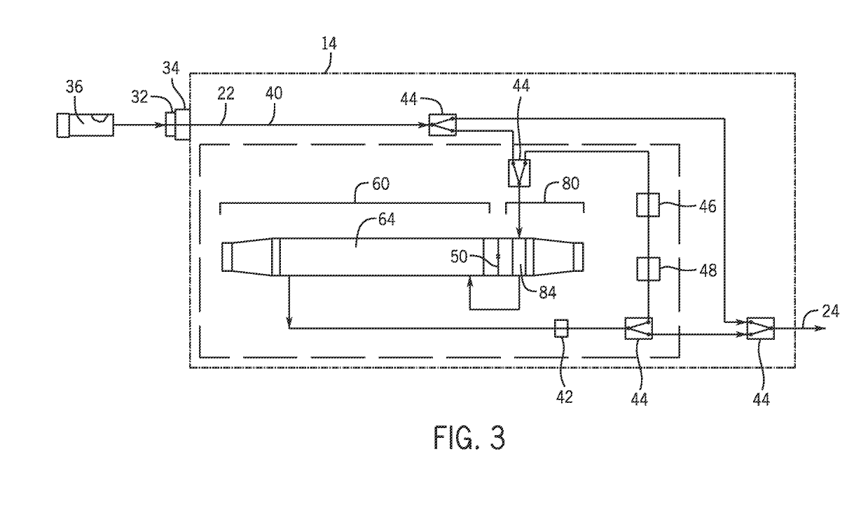

With reference now to FIG. 3 an alternative gas sample conduit arrangement is shown for guiding exhaled air into and through a housing 14. In this arrangement, the gas sample travels in series, rather than in parallel, through the second sample cell and then the first sample cell. This arrangement ensures a complete flushing of sample cells 64 and 84 without the need to balance the impedance along separate pathways to the cells.

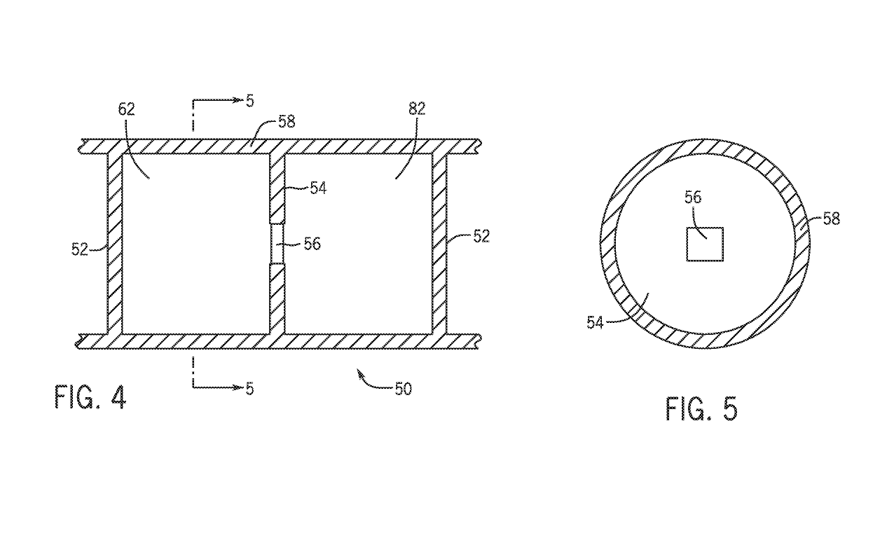

With reference to FIGS. 4 and 5, bi-directional correlation cell 50 is shown in greater detail. A transparent wall 52 separates sample cell 64 from a first side 62 of the correlation cell and opposing transparent wall 52 separates sample cell 84 from a second side 82 of the correlation cell so that most InfraRed radiation that is not absorbed by the gas in sample cells enters respective sides of the correlation cell. A pressure transducer 56, disposed along common opaque wall 54, generates an electrical response to differences in pressure between the front 62 and back 82 portions of correlation cell 50. When portion 62 of the correlation cell is illuminated, the portion 82 functions as its pressure reference, and conversely when the back 82 is illuminated the front 62 serves as the pressure.

With reference to FIG. 6 an embodiment of the electrical schematic 100 of the device 10 is further illustrated as including a power supply 110 and controller 120. Electrical conduits couple power supply 110 with flow meter 42 (G), pressure transducers 56 (H), valves 44 (I), radiation sources 70 and 90 (J), and pump 48 (K). The preferable power supply 110 is a rechargeable battery capable of delivering enough energy to keep the handheld device operating continuously at several Watt for hours.

Controller 120 has multiple data inputs and data outputs. The embodiment illustrated in FIG. 6 shows data inputs from flow meter 42 (A), pressure transducer 56 (B), and user control switch 20 (Start Test). Controller 120 also transmits output data signals to control switching of three way valves 44 (D), to continuous processing of information and output of corresponding information to display (16), and to clocking output 124 to the power supply 110 for periodic pulsing of the radiation sources 70 and 90. The displayed information is derived in part from the transducers' signals demodulated relative to the timing inferred from the system clock 124 which is also used to activate the radiation sources.

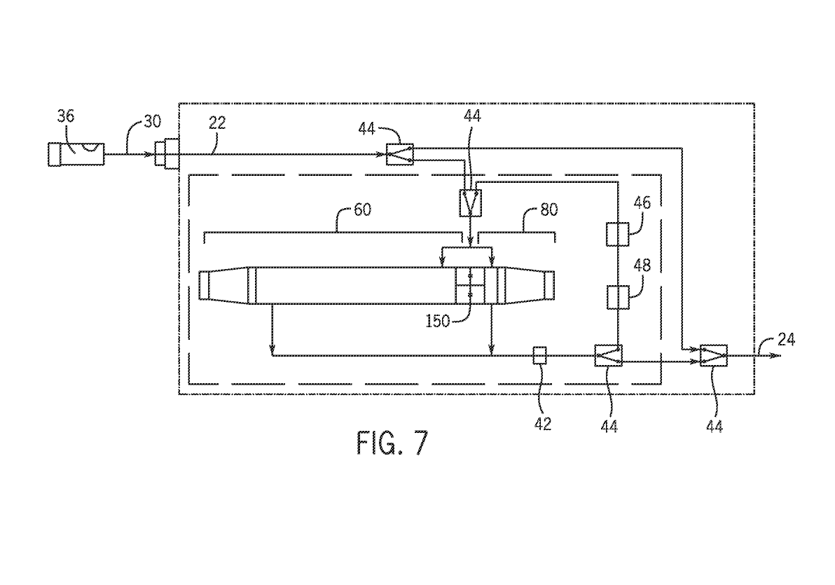

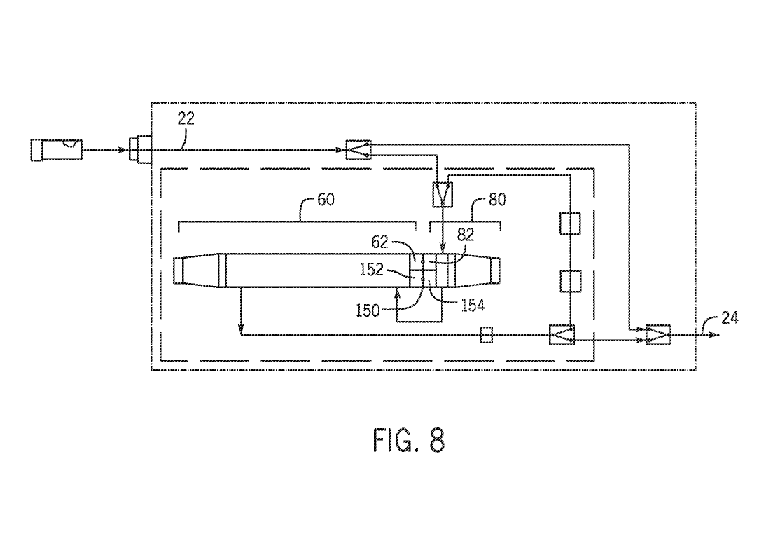

With reference to FIG. 7, an embodiment of the invention is shown. The device 10 is similar to the embodiments illustrated in FIG. 2 except an alternate dual bi-correlation cell 150 is shown coupled to probes 60 and 80. A first divided portion of the correlation cell 150 includes correlation cells 62 and 82 with a pressure transducer 56 positioned on opaque dividing wall 54. Cell portions 62 and 82 contain substantial concentrations of both CO2 isotopes and negligible concentrations of any possible interfering gases. The opposing cells 152 and 154 of the dual bi-direction cell 150 are the same size as portions 62 and 82 but contain trace gases distinct from the gas sample. For example, the trace gas may be selected to have negligible concentrations of gases found in human breath, while demonstrating an absorption of radiation per molecule that is comparable to the two isotopes of CO2. Correlation cell 150 can therefore serve to calibrate the relative strength of the two radiation streams incident on the sample cells 64 and 84. The resulting data output from the pressure transducer couple between portions 152 and 154 may serve to validate the calibration by zero air or may be utilized to substitute for zero air as the user may choose. FIG. 8 demonstrates an alternate alternative gas sample transmission path similar to the path described for FIG. 3 utilizing the above described dual bi-directional correlation cell 150.

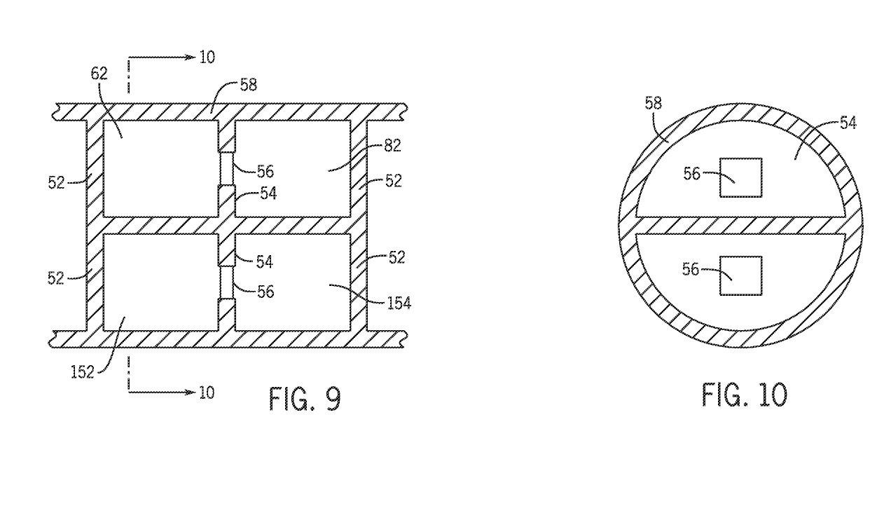

FIGS. 9 and 10 illustrates the dual bi-directional correlation cell 150 in greater detail. A transparent wall 52 separates sample cell 64 from a first side 62 and 152 of the correlation cell and opposing transparent wall 52 separates sample cell 84 and 154 from a second side 82 of the correlation cell so that most IR radiation that is not absorbed by the gas in sample cells enters respective sides of the correlation cell. Pressure transducers 56 are disposed along common opaque wall 54 between portions 62 and 82 and portions 152 and 154. The pressure transducers generate an electrical response to differences in pressure between the front 62, 152 and back 82, 154 portions of correlation cell 150. When portion 62 of the correlation cell is illuminated, the portion 82 functions as its pressure reference, and conversely when the back 82 is illuminated the front 62 serves as the pressure.

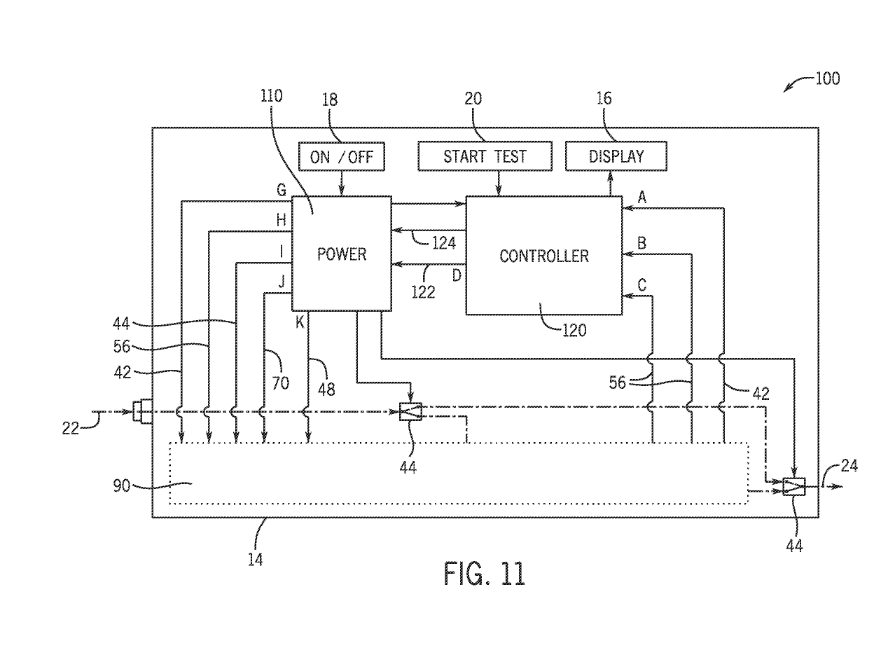

With reference to FIG. 11, an embodiment of the electrical schematic 100 of the device 10 is further illustrated as including a power supply 110 and controller 120. Electrical conduits couple power supply 110 with flow meter 42 (G), pressure transducers 56 (H), valves 44 (I), radiation sources 70 and 90 (J), and pump 48 (K). The preferable power supply 110 is a rechargeable battery capable of delivering enough energy to keep the handheld device operating continuously at several Watt for hours.

Controller 120 has multiple data inputs and data outputs. The embodiment illustrated in FIG. 11 shows data inputs from flow meter 42 (A), pressure transducers 56 (B) and (C), and user control switch 20 (Start Test). Controller 120 also transmits output data signals to control switching of three way valves 44 (D), to continuous processing of information and output of corresponding information to display (16), and to clocking output 124 to the power supply 110 for periodic pulsing of the radiation sources 70 and 90. The displayed information is derived in part from the transducers' signals demodulated relative to the timing inferred from the system clock 124 which is also used to activate the radiation sources.

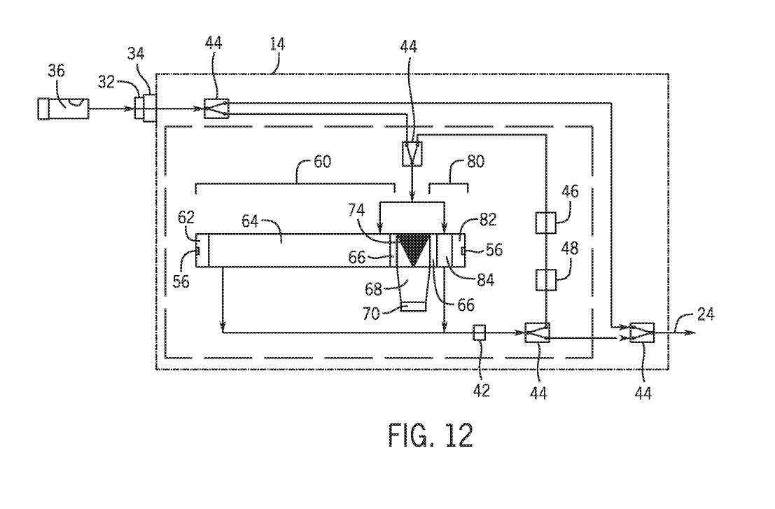

With reference to FIG. 12 and embodiment of the device 10 is shown that utilizes a single source of radiant energy. The stream of radiation from source 70 is collimated through optics 68 onto a beam splitter 74 that directs simultaneous streams of radiant energy through band pass filters 66 and into aligned probes 60 and 80. Probe 60 includes sample cell, 64, correlation cell 62 and pressure transducer 56. Probe 60 is sensitive to 13CO2 radiation. Similarly, probe 80 includes sample cell 84 and correlation cell 82. Probe 80 is sensitive to 12CO2. Calibration of the probes is similar to the above described calibration methods. The ratio of absorptions in the two correlation cells, corrected for their responses to zero air, serves as the needed measure of the ratio of concentrations of the two isotopes.

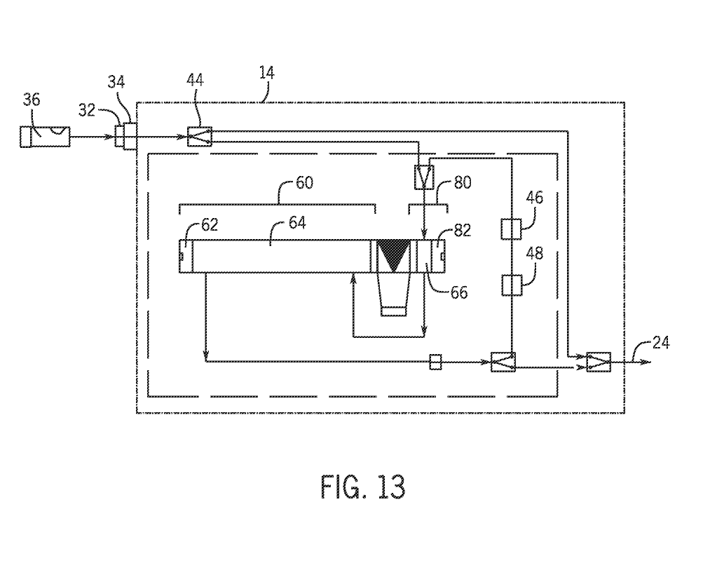

With reference to FIG. 13 an alternative gas sample conduit arrangement is shown for guiding exhaled air into and through a housing 14 similar to that shown in FIG. 12. In this arrangement, the gas sample travels in series, rather than in parallel, through the second sample cell and then the first sample cell. This arrangement ensures a complete flushing of sample cells 64 and 84 without the need to balance the impedance along separate pathways to the cells.

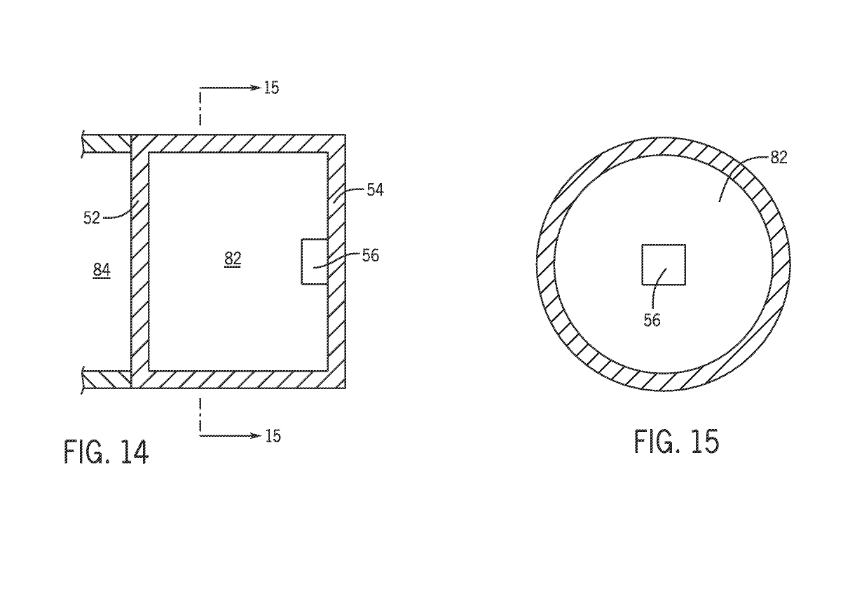

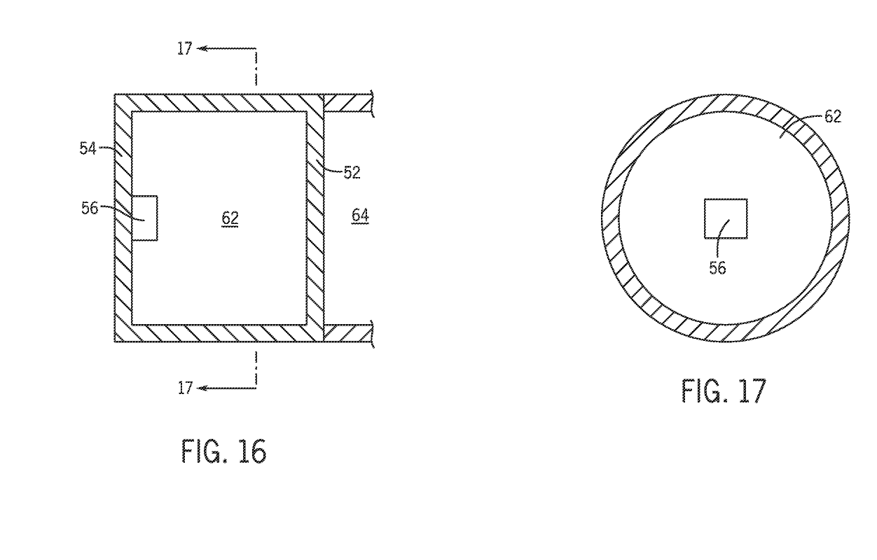

With reference to FIGS. 14-15 and 16-17 a split correlation cell 160 is illustrated in greater detail. Referring first to the correlation cell portion shown in FIGS. 16-17 a transparent wall 52 separates sample cell 64 from a first correlation portion 62 so that most IR radiation that is not absorbed by the gas in sample cell 64 enters the correlation cell 62. A pressure transducer 56, disposed along opaque wall 54, generates an electrical response to differences in pressure within the cell portion 62. Likewise, with reference to the correlation cell portion shown in FIGS. 14-15 a transparent wall 52 separates sample cell 84 from a second correlation portion 82 so that most IR radiation that is not absorbed by the gas in sample cell 84 enters the second portion 82. A pressure transducer 56, disposed along opaque wall 54, generates an electrical response to differences in pressure within the cell portion 82.

Pressure transducer 56 described above is preferably a MEMS devices of known suitable construction. Using batch fabrication and other techniques employed in the semiconductor industry, MEMS pressure transducers can be manufactured at low cost and with the appropriate size on silicon wafers. The correlation cell may be further manufactured using semiconductor processing techniques to create the correlation cells a useful internal volume having a select gas hermetically sealed within the correlation cell. Alternative a small aperture may be formed in the transparent wall 52 such that the internal volume can serve as the pressure reference for the correlation cell.

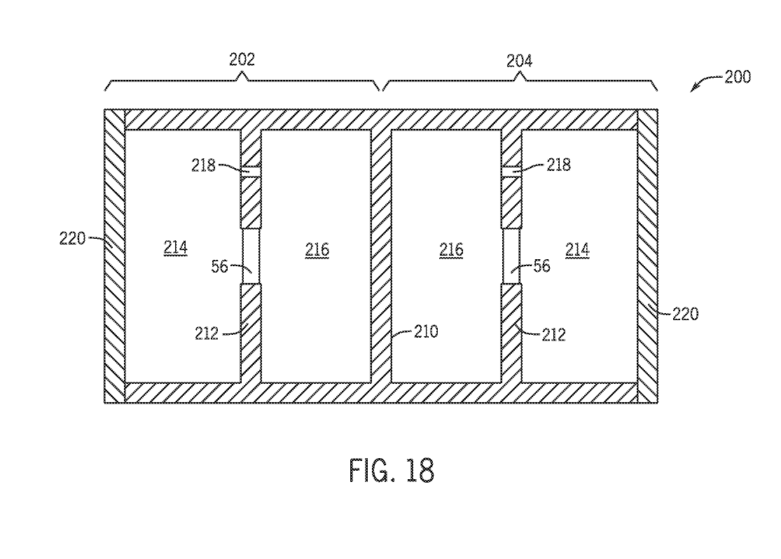

With reference to FIG. 18, an alternative correlation cell 200 is shown. The correlation cell 200 includes independent cell portions 202 and 204 that are oriented back-to-back and share a common opaque inert wall segment 210. Each cell portion may contain a pure sample of gas. For example, correlation cell 202 may contain 12CO2 and correlation cell 204 may contain 13CO2. Each cell portion is configured to include a dividing wall 212 that splits the cell into a radiation sensitive volume 214 and a pressure reference volume 216. A transparent wall segment 220 seals each end of the cell portion 202 and 204. A pressure transducer 56 is disposed along each dividing wall 212. A small aperture 218 extending through dividing wall 212 allows for gradual equalization of pressure on both sides of wall 212. The aperture 218 has high impedance to flow, enabling the pressure transducer 56 to respond to momentary pressure changes due to absorption of pulsed IR energy in radiation sensitive volumes 214.

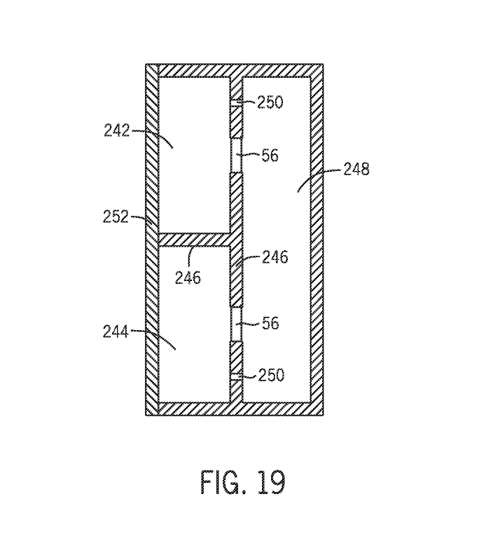

FIG. 19 presents another embodiment of a correlation cell of the invention. The configuration illustrated in FIG. 19 allows a configuration of probes 60 and 80 aligned side by side or in parallel rather than in series as above described. The correlation cell 240 may have a single reference volume and correlation portions 242 and 244 having a single gas contained therein. Yet another embodiment (not shown) would include separate reference volumes and separate pure samples of gas that are arranged to allow a parallel probe arrangement. Radiation enters by the transparent wall 384. The correlation cells 242 and 244 share a common opaque wall 246. Pressure transducers 56 sense pressure fluctuations caused by the absorption of radiation by the gas of the two correlation cells. A pair of small apertures 250 maintains zero mean pressure difference among all three volumes, the two correlation cells and the reference volume 248. The apertures have enough impedance to flow to enable the correlation cells to respond to the fast fluctuation of pressure coming from absorption of radiation. An opaque wall 398 bounds the device on the top, bottom and other side. In either of these embodiments a relative concentration of the isotopes, i.e. the isotopic ratio, is calculated based on the readings from the pairs of pressure transducers 56. Those skilled in the art will appreciate that the small apertures may be used to keep mean differential pressure at zero and may be incorporated into the other correlation cell configurations described above as desired and radiation filtering is appropriate.

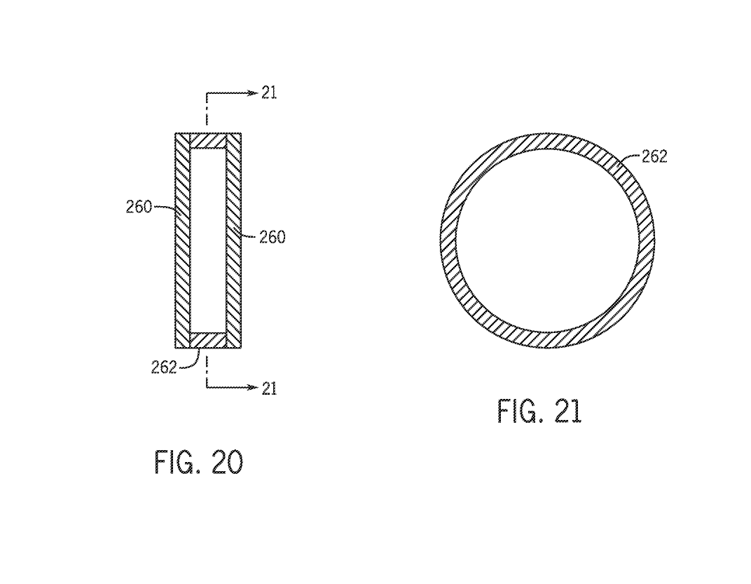

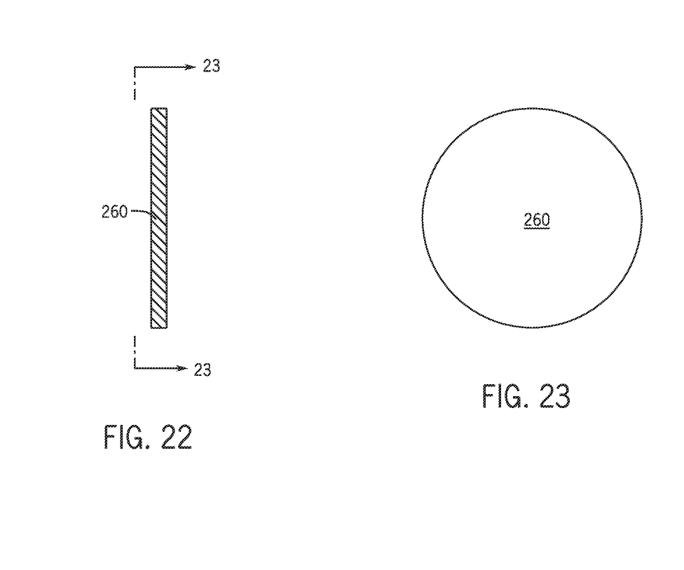

With reference to FIGS. 20-23, embodiments of band pass filters will be described in greater detail. FIGS. 20-21 shows a filter that uses isotopes to filter the radiation energy. A transparent disc material (e.g.; anti-reflection coated Al2O3) or narrow band filter disc material 260 is used to create a chamber containing a selected isotope to create an optical window that allows radiant energy to pass there through. Sidewalls 262 are constructed of a cylindrical opaque material. A sample of CO2 isotope can be captured as part of the filter's fabrication process, or it can be flushed through and then captured by one or more fill tubes (not shown) that can then be sealed by pinch-off or other hermetic mechanism. In use, a filter filled with pure sample of 12CO2 can be inserted into a 13CO2 probe to negate any of its residual sensitivity to the 12CO2 isotope. Likewise, a 13CO2 filter will negate the residual sensitivity of a 12CO2 probe to 13CO2 of the sample cell. This form of isotope filtering enhances the filtering inherent in the correlation cells that use the isotopes themselves as part of the overall detection mechanism. FIGS. 22-23 shows a narrowband filter disc 260 made of a material spanning the absorption bands of both CO2 isotopes.

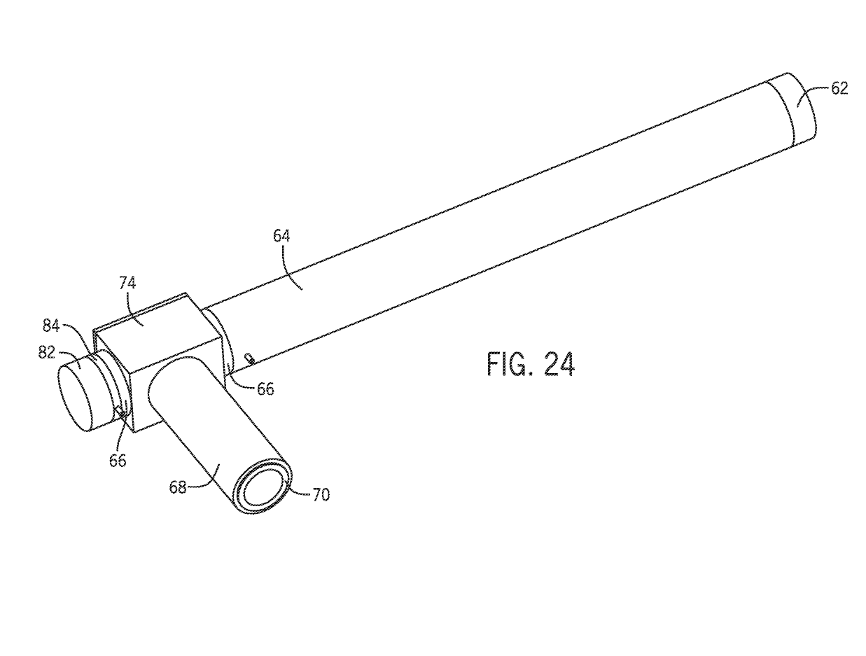

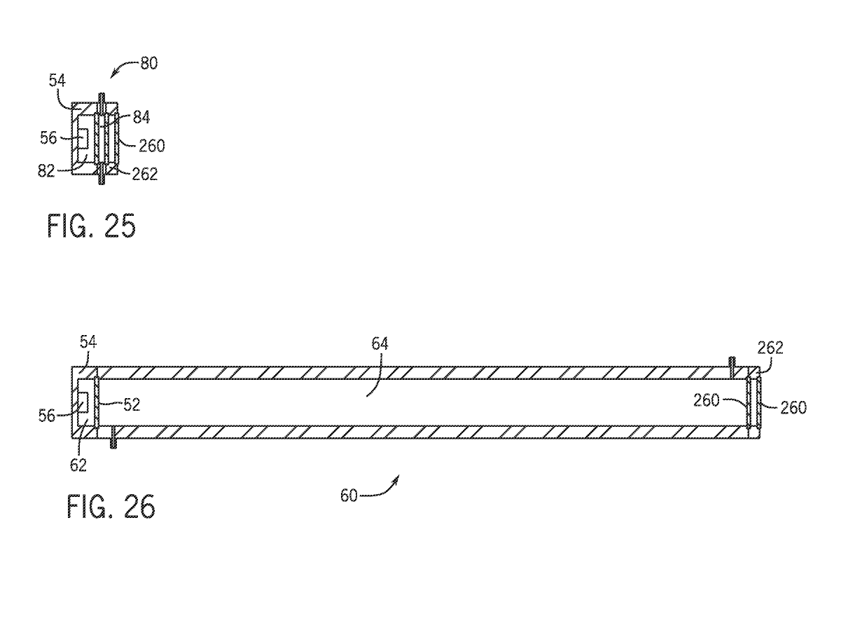

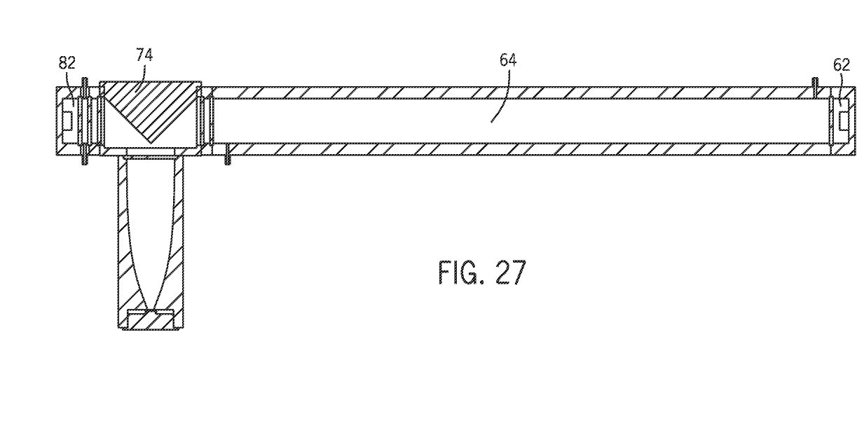

With reference to FIGS. 24-27 an embodiment of the device previously discussed with reference to FIG. 12 will described in further detail. Radiation from source 70 is collimated by collimator 68 onto beam splitter 74, sending radiation energy into correlation cells 62 and 82. FIGS. 24 and 27 depict a solid model and section of an integration of probe 80 illustrated in FIG. 25 and probe 60 illustrated in FIG. 26 integrated into a radiation source 70, collimator 68 and beam splitter 74. Alternatively, probes 60 and 80 may be modular and coupled into an assembly rather than in a fixed configuration. Probe 60 has its sample cell 64 bracketed by correlation cell 62 and isotope filter 260. Inlets route a gas sample through the sample cell. Probe 80 has its sample cell 84 bracketed by correlation cell 82 and isotope filter 260. Inlets route air through the sample cell 84. The pair of probes 60 and 80 with any of the above described modifications may be integrated with one or more radiation sources to complete the CO2 gas analysis device.

Having described the constructional features of the invention a method of using the invention will next be discussed. An advantage of device 10 is portability, due to its small size, low power requirement, low cost and ease of use. A test subject simply breathes into mouthpiece 36 to create a flow of exhaled air through the conduit arrangement and out of housing 14 through exit port 24. When air is provided to sample cells in parallel fashion as described in certain embodiments, it is important to match the impedance of the paths to the sample cells to ensure that both of the cells are filled with exhaled air as the test subject breaths into the mouthpiece. Three way valves are utilized to ensure this flow. A measurement cycle may involve a three-step sequence of automatic valve settings and responses to breath and zero air. The radiation sources are left on for the duration of the cycle. Initially, valves 44 route zero air through the device. When enough flow has been integrated by flow meter 42, responses of the correlation cells to zero air are recorded. Next all valves 44 are set to enable flow of breath sample through the sample cells. When the integrated flow is large enough, valves 44 are set for bypass flow. By action of the three way valves, the breath sample may be captured with the sample cells and can be analyzed. Responses to radiant energy by the correlation cells are recorded. Continued exhaled air, if present, exits through the housing via bypass conduit. The measurement cycle may end with a repeat of the response to zero air. Once the measurement cycle is complete, the responses to zero air is subtracted from the respective responses to breath to evaluate the ratio of the concentrations of CO2 isotopes of the breath sample.

Reduction of correlation cells' responses to ratio of isotopic concentrations is the same for all embodiments of device 10. Further, processing responsive signals are typically the same for zero air and breath samples, however, when a dual bi-directional cell is incorporated into the device 10, one of the correlation cells may be used to predict the response to zero air without the need for the preparation and processing of the zero air. In order to obtain an accurate ratio it is important to correct all correlation cell responses to zero air.

The processing of radiation by controller can be described with reference to the device described in conjunction with FIG. 1. With air captured from the same breath sample in each of the sample cells, power is provided out of phase to IR sources to direct infrared radiation through probe assemblies in opposite axial directions. The radiation energy passes through band pass filter, sample cell and then into correlation cell. The exhaled air in the sample cell absorbs part of the IR radiation of isotope 12CO2 from the source. Similarly, a portion of the IR radiation of isotope 13CO2 from the source is absorbed in sample cell. Opaque walls of the correlation cell prevent IR radiation generated by either source from entering the half of the bi-directional correlation cell associated with the other. A ratio of the responses of the correlation cell, when corrected for responses to zero air, serves as the desired measure of the 13CO2/12CO2 ratio of isotopes in the sample of breath.

The absorption of IR radiation in correlation cells increases the temperature and thus the pressure of their hermetically sealed samples of gas. In each correlation cell, the amount of the pressure increase is commensurate with the amount of absorbed IR radiation. A greater absorption of IR radiation in one of the cells leads to a greater pressure increase in that cell, creating a pressure between the cells detected by differential pressure transducers. The device of the present invention may also be utilized to detect concentrations of naturally occurring CO2 of the background atmosphere. Since absorption of IR radiation per molecule is the same for 12CO2 and 13CO2, the device of the invention has the ability, by action of filtering or adjustment of the contents of the correlation cells, to measure the isotopes separately.

Further, possible drift between separated components of the invention is compensated by design including operating protocols. For example, the ratio of radiation from two sources may be monitored by the processing of air stripped of all CO2. The same pressure transducer is then used for the detection of both isotopes, and there is no differential drift associated with it. When separate bi-directional correlation cells are filled with a trace gas (which is absent in significant levels in human breath and background atmosphere) the controller may be used to detect the levels of radiation incident on the samples of breath. The trace gas has insignificant spectroscopic overlap with either isotope of CO2. This action provides a second or substitute measure of the response to zero air. As a further example, radiation from a single source may be split in two, thereby negating the effect of any drift in the ratio of the incident radiation streams. When correlation cells are separated by a substantial distance, drift between the radiation sources is expected to be a larger factor, but, if necessary, drift between the correlation cells can be evaluated by the application of zero air.

By using multiple variations of the present invention, the absorptions of infrared radiation in correlation cells containing pure samples of the 12CO2 isotope or 13CO2 isotopes, or some combination of the two, are compared to yield relative concentration information concerning the two isotopes. The absorption of IR radiation is detected preferably by sensing the momentary changes in pressure of the gas contained in each correlation cell as pulsed IR radiation is absorbed by its gas. This allows the use of MEMS technology including pressure transducers in lieu of a typical sensor of radiation. As a further refinement, a single MEMS transducer between adjacent correlation cells can measure the differential pressure and thereby directly indicate relative concentration information in the form of an isotopic ratio.

These and various other aspects and features of the invention are described with the intent to be illustrative, and not restrictive. This invention has been described herein with detail in order to comply with the patent statutes and to provide those skilled in the art with information needed to apply the novel principles and to construct and use such specialized components as are required. It is to be understood, however, that the invention can be carried out by specifically different constructions, and that various modifications, both as to the construction and operating procedures, can be accomplished without departing from the scope of the invention. Further, in the appended claims, the transitional terms comprising and including are used in the open ended sense in that elements in addition to those enumerated may also be present. Other examples will be apparent to those of skill in the art upon reviewing this document.

What is claimed is:

1. A device for determining concentrations of a selected isotope in a gas, said device comprising:
an air intake;
sample cells adapted to receive an air sample;
correlation cells having hermetically sealed gas chambers therein, said correlation cells including a first correlation cell having 12CO2 isotopes of carbon dioxide gas and a second correlation cell having 13CO2 isotopes of carbon dioxide gas;
radiant energy sources;
an isotopic analyzer;
an air outtake; and
air conduits coupling said air intake, sample cells and air outtake.

2. The device according to claim 1, further including a housing containing the sample cells, correlation cells, radiant energy sources, isotopic analyzer and air conduits.

3. The device according to claim 1, wherein the correlation cells are bi-directional.

4. The device according to claim 1, wherein said radiant energy sources includes a single radiant energy generator and a beam splitter that directs radiant energy towards separate sample cells and correlation cells.

5. The device according to claim 1, further including collimating optics coupled with the radiant energy sources.

6. The device according to claim 1, wherein the sample cells and correlation cells are aligned in series.

7. The device to claim 1, further including a desiccant filter coupled in series between the air intake and sample cells.

8. The device according to claim 1, further including a valve, flow meter, and pumps coupled to the air conduits to purge the sample cell.

9. The device according to claim 1, wherein said radiant energy sources are controlled to transmit radiant energy at selected bandwidths for desired absorption by select gases.

10. A device for determining relative concentrations of a plurality of isotopes of a gas in a gas sample, including:
a first sample cell adapted to receive a first portion of a gas sample comprising a selected gas;
a second sample cell adapted to receive a second portion of the gas sample;
a first correlation cell containing a first gas comprising a first isotope of the selected gas while being substantially free of a second isotope of the selected gas;
a second correlation cell containing a second gas comprising the second isotope while being substantially free of the first isotope;
a radiant energy source adapted to direct pulsed radiant energy along a first path through the first sample cell and into the first correlation cell, and further adapted to direct pulsed radiant energy along a second path through the second sample cell and into the second correlation cell; and
a sensing component operatively associated with the first and second correlation cells, responsive to absorption of radiant energy in the first correlation cell by the first gas at a first absorption level and further responsive to absorption of radiant energy in the second correlation cell by the second gas at a second absorption level, and adapted to compare the first and second absorption levels to generate an indication of relative concentration of the first isotope and the second isotope in the gas sample.

11. The device of claim 10, further including a calibration component, wherein the radiant energy source comprises a first IR source proximate the first sample cell and a second IR source proximate the second sample cell, wherein the calibration component is adapted to compensate for a difference in amplitude between the first and second IR sources, if any.

12. The device of claim 11, wherein the first correlation cell is joined to the first sample cell to facilitate a linear propagation of IR energy through the first sample cell into the first correlation cell;
the second correlation cell is joined to the second sample cell to facilitate a linear propagation of IR energy through the second sample cell into the second correlation cell; and
the first and second correlation cells are joined along a common wall that isolates each of the correlation cells from the other.

13. The device of claim 12, wherein the sample cells and the correlation cells are arranged linearly to provide for said linear propagation of IR energy in a first direction through the first sample cell into the first correlation cell and in a second, opposite direction through the second sample cell into the second correlation cell.

14. The device of claim 13, wherein the calibration component comprises a first gas-containing calibration cell disposed proximate the first correlation cell and a second gas-containing calibration cell disposed proximate the second correlation cell, wherein the sensing component further is operatively associated with the first and second calibration cells and adapted to compare respective third and fourth levels of absorption of IR energy in the first and second calibration cells.

15. The device of claim 10, wherein the sensing component comprises a pressure transducing component adapted to detect a difference in pressure between the first correlation cell and the second correlation cell to generate the indication of relative concentration.

16. The device of claim 15, wherein the first and second correlation cells are joined to one another along a common wall, and the pressure transducing component comprises a pressure transducer disposed along the common wall.

17. The device of claim 15, wherein the first correlation cell is joined to the first sample cell to share a first common wall with the first sample cell, and the second correlation cell is joined to the second sample cell to share a second common wall with the second sample cell; and
the pressure transducing component comprises a first pressure transducer disposed along the first common wall and a second pressure transducer disposed along the second common wall.

18. The device of claim 10, wherein the first isotope constitutes at least ten percent of the first gas by volume, and the second isotope constitutes at least ten percent of the second gas by volume.

19. The device of claim 18, wherein the first gas consists essentially of the first isotope, and the second gas consists essentially of the second isotope.

20. The device of claim 10, further including first and second narrow band pass filters disposed at respective first and second entrance ends of the first and second sample cells, for confining the pulsed radiant energy to a predetermined radiant energy bandwidth selected for absorption by the selected gas.

21. The device of claim 20, wherein the selected gas is carbon dioxide, the first isotope is 12CO2, and the second isotope is 13CO2.

22. The device of claim 10, wherein the radiant energy source comprises an incandescent filament operable to modulate an amplitude and frequency of the radiant energy.

23. The device of claim 10, further including a conduit arrangement for simultaneously conducting the first and second portions of the gas sample into the first and second sample cells, respectively.

24. A device for determining a selected concentration of a targeted isotope in a breath of air, said device including,
a first sample cell adapted to receive and contain a first portion of a breath sample;
a second sample cell adapted to receive and contain a second portion of the breath sample;
a first correlation cell containing a first gas that comprises a first isotope of a selected gas while being substantially free of a second isotope of the selected gas;
a second correlation cell containing a second gas that comprises the second isotope of the selected gas while being substantially free of the first isotope;
a radiant energy source adapted to direct pulsed radiant energy along a first path through the first sample cell and into the first correlation cell, and further adapted to direct pulsed radiant energy along a second path through the second sample cell and into the second correlation cell; and
a sensing component operatively associated with the first and second correlation cells, adapted to compare a first level of absorption of radiant energy by the first gas in the first correlation cell with a second level of absorption of the radiant energy by the second gas in the second correlation cell, to generate an indication of relative concentration of the first isotope and the second isotope in the breath sample.

25. The analyzer of claim 24, further including a housing containing the sample cells, the correlation cells, the radiant energy source and the sensing component; and
a conduit arrangement accessible outside of the housing for conducting the first and second portions of the breath sample from outside of the housing to the first and second sample cells, respectively.

26. The analyzer of claim 25, wherein the conduit arrangement includes a bypass conduit adapted to shunt breath past the first and second sample cells after the cells have respectively received the first and second portions of the breath sample.

27. The analyzer of claim 25, wherein the conduit arrangement comprises a first conduit segment for providing the first portion of the breath sample to the first sample cell, and a second conduit segment for providing the second portion of the breath sample to the second sample cell, and the first and second conduit segments have substantially the same impedance to facilitate a simultaneous flow of the first and second portions of the breath sample into the first and second sample cells, respectively.

28. The analyzer of claim 24, wherein the sample cells are integrally coupled, and arranged with the first and second correlation cells adjacent one another and the first and second sample cells relatively remote from one another, whereby the radiant energy directed along the first path and the radiant energy directed along the second path travel in opposite directions toward a junction of the correlation cells.

29. The analyzer of claim 24, wherein the radiant energy source comprises a first IR source for directing pulsed IR energy along the first path through the first sample cell, and a second IR source for directing pulsed IR energy along the second path through the second sample cell.

30. The analyzer of claim 29, further including a calibration component adapted to compensate for a difference in amplitude between the first and second IR sources.

31. The analyzer of claim 24, wherein the sensing component comprises a pressure transducer to detect a difference in pressure between the first correlation cell and the second correlation cell to generate the indication of relative concentration.

32. The analyzer of claim 31, wherein the first and second correlation cells are joined to one another along a common wall, and the pressure transducer is disposed along the common wall shared by the first and second correlation cells.

33. The analyzer of claim 32, wherein the first correlation cell is joined to the first sample cell to share a first common wall with the first sample cell, the second correlation cell is joined to the second sample cell to share a second common wall with the second sample cell; and
the pressure transducer comprises a first pressure transducer disposed along the first common wall, and a second pressure transducer disposed along the second common wall.

34. The analyzer of claim 33, wherein the first isotope constitutes at least ten percent of the first gas by volume, and the second isotope constitutes at least ten percent of the second gas by volume.

35. The analyzer of claim 34 wherein, the first gas consists essentially of the first isotope, and the second gas consists essentially of the second isotope.

36. The analyzer of claim 24, further including first and second narrow band filters disposed at respective first and second entrance ends of the first and second sample cells for confining the pulsed radiant energy to a predetermined radiant energy bandwidth selected for absorption by the selected gas.

37. The analyzer of claim 24, wherein the selected gas is carbon dioxide, the first isotope is 12CO2, and the second isotope is 13CO2.

38. The analyzer of claim 24, wherein the radiant energy source comprises an incandescent filament operable to modulate an amplitude and frequency of the radiant energy.

39. A process for determining relative concentrations of isotopes of a gas in a breath sample, including:
directing pulsed radiant energy along a first path through a first portion of a breath sample and into a first correlation cell containing a first gas, wherein the first gas comprises a first isotope of a selected gas and is substantially free of a second isotope of the selected gas;
directing pulsed radiant energy along a second path through a second portion of the breath sample and into a second correlation cell containing a second gas, wherein the second gas comprises the second isotope and is substantially free of the first isotope; and
sensing a difference in pressure between the first correlation cell and the second correlation cell to generate an indication of relative concentration of the first and second isotopes in the breath sample.
